(19) United States
(12) Patent Application Publication
(10) Pub. No.: US 2017/0027951 A1
(43) Pub. Date: Feb. 2, 2017

(54) METHODS AND COMPOSITIONS TO TREAT CANCERS INVOLVING EGFR

(71) Applicant: Southern Research Institute, Birmingham, AL (US)

(72) Inventor: Lidija Klampfer, Birmingham, AL (US)

(21) Appl. No.: 15/205,894

(22) Filed: Jul. 8, 2016

Related U.S. Application Data
(60) Provisional application No. 62/197,454, filed on Jul. 27, 2015.

(51) Int. Cl. A61K 31/5377; A61K 31/166; A61K 45/06; A61K 31/5025

(52) U.S. Cl. CPC A61K 31/5377; A61K 31/5025; A61K 31/166; A61K 45/06

(57) ABSTRACT

In one aspect, the invention relates to pharmaceutical compositions and methods for treating a cancer comprising administration of an EGFR inhibitor and a MEK or MET inhibitor. In various aspects, the methods can comprise determination of increased levels of HGF, e.g., determining if a patient has an increased level of serum HGF. This abstract is intended as a scanning tool for purposes of searching in the particular art and is not intended to be limiting of the present invention.

### CROSS-REFERENCE TO RELATED APPLICATIONS

This application claims the benefit of U.S. Provisional Application No. 62/197,454, filed on Jul. 27, 2015, which is incorporated herein by reference in its entirety.

### BACKGROUND

A significant advance in the treatment of cancer has been the introduction of targeted cancer therapeutic agents such as epidermal growth factor receptor (“EGFR”) inhibitors. For example, the survival rate of patients with metastatic colorectal cancer (“mCRC”) has significantly improved in recent years due to availability of the monoclonal antibodies, cetuximab (Erbitux, Bristol Myers Squibb, Princeton, N.J.) and panitumumab (Vectibix®, Amgen, Thousand Oaks, Calif.) (see Amado R. G., et al., J. Clin. Oncol. (2008) 26:1626-34; Jonker D. J., et al., New Engl. J. Med. (2007) 357:2040-8). Large randomized trials established that KRas mutations predict the lack of clinical benefit in response to anti-EGFR therapy in patients with mCRC. Based on such data, the US Food and Drug Administration (“USFDA”) recommended that the use of the anti-EGFR monoclonal antibodies be restricted to patients with a wild-type KRas (“WT KRas”) gene status (see Tejpar S. et al., Oncologist (2010) 15:390-404; Allegra C. J. et al., J. Clin. Oncol. (2009) 27:2091-6). However, only a small fraction of WT KRas patients responds to EGFR inhibitors EGFR (“EGFRi”). For example, only 17% of KRas WT patients benefit from panitumumab (see Amado R. G., et al., J. Clin. Oncol. (2008) 26:1626-34) and 12.8% of the patients with WT KRas respond to cetuximab (see Karapetis C. S., et al., New Engl. J. Med. (2008) 359:1757-65).

Thus, despite advances in research directed to improved cancer treatment, in particular therapeutic agents targeting EGFR, there is still a scarcity of compounds that are effective in treating certain cancer populations with EGFR inhibitors. Moreover, there are no established biomarkers for predicting which patient populations can benefit from therapeutic agents targeting EGFR. These needs and other needs are satisfied by the present invention.

### SUMMARY

In accordance with the purpose(s) of the invention, as embodied and broadly described herein, the invention, in one aspect, relates to methods of treating cancer with EGFR inhibitors. In various aspects, the invention pertains to combination therapies comprising at least one EGFR inhibitor used in combination with a MET kinase inhibitor. In a further aspect, invention pertains to combination therapies comprising at least one EGFR inhibitor used in combination with a MEK kinase inhibitor.

Disclosed are pharmaceutical compositions comprising: (a) an effective amount of an EGFR inhibitor, or a pharmaceutically acceptable salt thereof; and (b) an effective amount of a MEK inhibitor or a MET inhibitor, or a combination thereof, or a pharmaceutically acceptable salt thereof; and a pharmaceutically acceptable carrier.

Also disclosed are methods for the treatment of a cancer in a mammal, the methods comprising the step of co-administering to the mammal: (a) an effective amount of an EGFR inhibitor, or a pharmaceutically acceptable salt thereof; and (b) an effective amount of a MEK inhibitor or a MET inhibitor, or a combination thereof, or a pharmaceutically acceptable salt thereof.

Also disclosed are methods for the treatment of colorectal cancer, the method comprising the step of administering to the human: (a) an effective amount of an EGFR inhibitor, or a pharmaceutically acceptable salt thereof; and (b) an effective amount of a MEK inhibitor or a MET inhibitor, or a combination thereof, or a pharmaceutically acceptable salt thereof.

Also disclosed are methods for the treatment of a cancer, the method comprising the steps of: identifying a mammal with increased HGF levels; and administering the mammal with increased HGF levels: (a) an effective amount of an EGFR inhibitor, or a pharmaceutically acceptable salt thereof; and (b) an effective amount of a MEK inhibitor or a MET inhibitor, or a combination thereof, or a pharmaceutically acceptable salt thereof.

Also disclosed are methods for decreasing resistance of a cancer to treatment with an EGFR inhibitor, the method comprising the steps of: (a) identifying a patient with increase HGF expression; and (b) administering to the patient with increased HGF expression a MET inhibitor.

Also disclosed are kits comprising: (a) an effective amount of an EGFR inhibitor, or a pharmaceutically acceptable salt thereof; and (b) an effective amount of a MEK inhibitor or a MET inhibitor, or a combination thereof, or a pharmaceutically acceptable salt thereof.

Also disclosed are medicaments comprising the disclosed pharmaceutical compositions or disclosed combinations of therapeutic agents.

Also disclosed are uses comprising the disclosed pharmaceutical compositions or disclosed combinations of therapeutic agents.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIGS. 1A-1D show representative data for the effect on EGFR inhibitors on cancer cells with a wild-type KRas gene status (“WT KRas”). RKO cells (FIGS. 1A, 1B, and iD), which are WT KRas and express HGF, or HT29 cells (FIG. 1C), which do not express HGF, were treated with gefitinib (5 μM) in the absence or presence of a MET kinase inhibitor (JNJ 38877605; 1 μM) or a MEKI kinase inhibitor (PD184161; 0.01 μM, 0.05 μM, or 1 μM) as indicated. FIG. 1A shows the effect of the indicated treatments on RKO cell viability at 96 hours after treatment. FIG. 1B shows the effect of the indicated treatments on RKO cell colony formation at 10 days after treatment. FIG. 1C shows the effect of the indicated treatments on HT29 cell viability at 96 hours after treatment. FIG. 1D shows the effect of the gefitinib treatment dose or times post-treatment on the phosphorylation status of ERK 1/2 in RKO cells.

FIGS. 2A-2B show representative data for the effect on EGFR inhibitors on cancer cells with a mutant KRas gene status (“MT KRas”). HCT116 cells, which are MT KRas, were treated with gefitinib (5 μM) in the absence or presence of a MET kinase inhibitor (JNJ 38877605; 1 μM) or a MEKI kinase inhibitor (PD184161; 0.01 μM or 0.02 μM) as indicated. FIG. 2A shows the effect of the indicated treatments on cell viability at 96 hours after treatment. FIG. 2B shows the effect of the indicated treatments on colony formation at 10 days after treatment.

FIGS. 3A-3C show representative data for the effect of HGF/MET signaling on fibroblast-mediated, HGF-dependent resistance to EGFR inhibitors. FIG. 3A shows clonogenic growth data for Caco2 cells treated with cetuximab (50 μg/ml) in the absence or presence of a MET kinase inhibitor (JNJ 38877605; 1 μM) as indicated at 12 days post-treatment. “FIB” indicates that the Caco2 cells were cultured in the presence of fibroblast conditioned medium. “CTRL” indicates untreated cells; #: p<0.05, compared to CTRL treatment; NS: not significant. FIG. 3B shows colony formation data obtained from Caco2 cells treated with gefitinib (1 μM) in the absence or presence of a MET kinase inhibitor (JNJ 38877605; 1 μM) as indicated at 10 days post-treatment. “FIB” indicates that the Caco2 cells were cultured in the presence of fibroblast conditioned medium. FIG. 3C shows caspase 3/7 activity data obtained from DiFi cells treated with gefitinib (1 μM) in the absence or presence of a MET kinase inhibitor (JNJ 38877605; 1 μM) as indicated at 18 hr post-treatment. “FIB” indicates that the Caco2 cells were cultured in the presence of fibroblast conditioned medium.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples included therein.

It is also to be understood that the terminology used herein is for the purpose of describing particular aspects only and is not intended to be limiting. As used in the specification and in the claims, the term “comprising” can include the aspects “consisting of” or “consisting essentially of.” Thus, for example, an aspect such as “a composition comprising A, B, and C” also includes aspects such as “a composition consisting of A, B, and C” and “a composition consisting essentially of A, B, and C.”

As used herein, the terms “by”, “comprising,” “comprises”, “comprised of,” “including,” “includes,” “included,” “involving,” “involves,” “involved,” and “such as” are used in their open, non-limiting sense.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### A. DEFINITIONS

Unless defined otherwise, all technical and scientific terms used herein have the same meaning as commonly understood by one of ordinary skill in the art to which this invention belongs. In this specification and in the claims which follow, reference will be made to a number of terms which shall be defined herein.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

The term “about” as used herein means approximately or near or around. For example, when the term “about” is used in relation to a specified dosage amount or range, the term “about” indicates that the dosage amount or range specified is an approximate dosage amount or range and that it includes not only the amount or range actually specified, but those amounts or ranges that may also be safe and effective amounts that are somewhat outside the cited amount or range. In some cases, “about” can mean+10% of the value modified by “about,” e.g., “about 100 mg” can be understood to include amounts from 90 mg to 110 mg, inclusive.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “colorectal cancer” is meant to include the well-accepted medical definition that defines colorectal cancer as a medical condition characterized by cancer of cells of the intestinal tract below the small intestine (e.g., the large intestine (colon), including the cecum, ascending colon, transverse colon, descending colon, and sigmoid colon, and rectum). Additionally, as used herein, the term “colorectal cancer” is meant to further include medical conditions which are characterized by cancer of cells of the duodenum and small intestine (jejunum and ileum).

As used herein, the term “metastasis” is meant to refer to the process in which cancer cells originating in one organ or part of the body relocate to another part of the body and continue to replicate. Metastasized cells subsequently form tumors which may further metastasize. Metastasis thus refers to the spread of cancer from the part of the body where it originally occurs to other parts of the body. As used herein, the term “metastasized colorectal cancer cells” is meant to refer to colorectal cancer cells which have metastasized; colorectal cancer cells localized in a part of the body other than the duodenum, small intestine (jejunum and ileum), large intestine (colon), including the cecum, ascending colon, transverse colon, descending colon, and sigmoid colon, and rectum.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

In some aspects of the disclosed methods, the subject has been diagnosed with a need for treatment of a cancer prior to the administering step. In some aspects of the disclosed methods, the subject has been diagnosed with a need for inhibiting EGFR activity prior to the administering step. In some aspects of the disclosed methods, the subject has been identified with an infectious disease that is treatable by inhibiting EGFR activity prior to the administering step. In some aspects of the disclosed methods, the subject has been identified as having an elevated serum HGF level or a mutation in the HGF gene associated with increased expression of HGF prior to the administering step. In one aspect, a subject can be treated prophylactically with a compound or composition disclosed herein, as discussed herein elsewhere.

As used herein, the terms “treat(s)”, “treated”, “treating” or “treatment” are used herein interchangeably and refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder; and supportive treatment, that is, treatment employed to supplement another specific therapy directed toward the improvement of the associated disease, pathological condition, or disorder. The term refer to any treatment of a disorder in a subject diagnosed or afflicted with such disorder and includes, but is not limited to (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with a cancer treatable by inhibiting EGFR activity” means having been subjected to a physical examination by a person of skill, for example, a physician, and found to have a condition that can be diagnosed or treated by a compound or composition that can inhibit EGFR activity. As a further example, “diagnosed with a need for treatment of cancer” refers to having been subjected to a physical examination by a person of skill, for example, a physician, and found to have a condition characterized by aberrant growth cells. For example, a subject can be diagnosed as having a cancer using suitable imaging techniques, or a combination of imaging techniques, known to a radiologist or an oncologist such as magnetic resonance imaging (MRI), positron emission topography (PET), or functional MRI (fMRI). These techniques can be used along with techniques such as histological analysis of biopsy samples.

As used herein, the phrase “identified to be in need of treatment of a cancer,” or the like, refers to selection of a subject or a patient based upon need for treatment of the cancer. For example, a subject or patient can be identified as having a need for treatment of a cancer (e.g., a cancer associated with EGFR or with EGFR and elevated expression of HGF) based upon an earlier diagnosis by a person of skill and thereafter subjected to treatment for the cancer. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14th edition), the Physicians' Desk Reference (64th edition), and The Pharmacological Basis of Therapeutics (12th edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term therapeutic agent also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

As used herein, the term “pharmaceutically acceptable non-toxic acids”, includes inorganic acids, organic acids, and salts prepared therefrom, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Preferred are citric, hydrobromic, hydrochloric, maleic, phosphoric, sulfuric, and tartaric acids.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### B. EGFR INHIBITORS

The present invention pertains to the use of EGFR inhibitors. In various aspects, the EGFR inhibitor is a chemical compound or small molecule inhibitor. In a further aspect, the EGFR inhibitor is an antibody therapeutic agent, e.g., a humanized monoclonal antibody.

In a further aspect, the invention pertains to use of an effective amount of an EGFR inhibitor. In various aspects, an “effective amount” can be a therapeutically effective amount. In a further aspect, an “effective amount” can be a prophylactically effective amount.

In a further aspect, the EGFR inhibitor is gefitinib, erlotinib, afatinib, rociletinib, vandetanib, lapatinib, dacomitinib, neratinib, icotinib, ibrutinib, panitumumab, zalutumumab, nimtuzumab, or matuzumab, or combinations thereof. In a still further aspect, the EGFR inhibitor is gefitinib, erlotinib, afatinib, lapatinib, dacomitinib, neratinib, cetuximab, or panitumumab, or combinations thereof.

In a further aspect, the EGFR inhibitor is gefitinib, erlotinib, afatinib, rociletinib, vandetanib, lapatinib, dacomitinib, neratinib, icotinib, ibrutinib, AZD9291, WZ4002, CO-1686, APC6113, PKC412, or HM61713, or a pharmaceutically acceptable thereof. In a further aspect, the EGFR inhibitor is gefitinib, erlotinib, vandetanib, or lapatinib, dacomitinib, neratinib, or a pharmaceutically acceptable thereof. In a still further aspect, the EGFR inhibitor is gefitinib, or a pharmaceutically acceptable thereof. In a yet further aspect, the EGFR inhibitor is erlotinib, or a pharmaceutically acceptable thereof. In a still further aspect, the EGFR inhibitor is vandetanib, or a pharmaceutically acceptable thereof. In an even further aspect, the EGFR inhibitor is lapatinib, dacomitinib, neratinib, or a pharmaceutically acceptable thereof.

In a further aspect, the EGFR inhibitor is cetuximab, panitumumab, zalutumumab, nimtuzumab, or matuzumab. In a still further aspect, the EGFR inhibitor is cetuximab or panitumumab. In a yet further aspect, the EGFR inhibitor is cetuximab. In an even further aspect, the EGFR inhibitor is panitumumab.

In various aspects, the EGFR inhibitor is used in combination with or co-administered with a MET inhibitor or a MEK inhibitor. In a further aspect, an effective amount of the EGFR inhibitor is used in combination with or co-administered with an effective amount of a MET inhibitor or an effective amount of a MEK inhibitor.

In a further aspect, the EGFR inhibitor is gefitinib, erlotinib, lapatinib, dacomitinib, neratinib, vandetanib, cetuximab, or panitumumab, or combinations thereof; and wherein the MEK inhibitor is trametinib. In a still further aspect, the EGFR inhibitor is gefitinib, erlotinib, lapatinib, dacomitinib, neratinib, vandetanib, cetuximab, or panitumumab, or combinations thereof; and wherein the MEK inhibitor is trametinib, binimetinib (MEK162), cobimetinib (GDC-0973), selumetinib (AZD6244), or PD184161, or combinations thereof. In a yet further aspect, the EGFR inhibitor is gefitinib, erlotinib, lapatinib, dacomitinib, neratinib, vandetanib, cetuximab, or panitumumab, or combinations thereof; and wherein the MEK inhibitor is trametinib. In an even further aspect, the EGFR inhibitor is gefitinib, erlotinib, lapatinib, dacomitinib, neratinib, vandetanib, cetuximab, or panitumumab, or combinations thereof; and wherein the MEK inhibitor is PD184161. In a still further aspect, the EGFR inhibitor is gefitinib; and wherein the MEK inhibitor is PD184161. In a yet further aspect, the EGFR inhibitor is gefitinib; and wherein the MEK inhibitor is trametinib.

In a further aspect, the EGFR inhibitor is gefitinib, erlotinib, lapatinib, dacomitinib, neratinib, vandetanib, cetuximab, or panitumumab, or combinations thereof; and wherein the MET inhibitor is crizotinib, cabozantinib, tivantinib, foretinib, golvatinib, or JNJ-38877605, or combinations thereof. In a still further aspect, the EGFR inhibitor is gefitinib; and wherein the MET inhibitor is JNJ-38877605.

In various aspects, the EGFR inhibitor is used in combination with or co-administered with a MET inhibitor or a MEK inhibitor, and one or more therapeutic agents known to treat cancer. In a further aspect, the EGFR inhibitor is used in combination with or co-administered with a MET inhibitor or a MEK inhibitor, and 5-fluorouracil, oxaliplatin, or leucovorin, or a combination thereof.

Methods of preparing each of the disclosed EGFR inhibitors are known in to the skilled artisan.

### C. MET INHIBITORS

The present invention pertains to the use of MET inhibitors. In various aspects, the MET inhibitor is crizotinib, cabozantinib, tivantinib, foretinib, golvatinib, JNJ-38877605, PHA-665752, SU11274, SGX-523, PF-04217903, EMD 1214063, INCB28060, MK-2461, NVP-BVU972, AMG458, BMS 794833, BMS 777607, MGCD-265, AMG-208, or BMS-754807, or combinations thereof. In a further aspect, the MET inhibitor is crizotinib, cabozantinib, tivantinib, foretinib, golvatinib, or JNJ-38877605. In a still further aspect, the MET inhibitor is crizotinib, cabozantinib, tivantinib, foretinib, or golvatinib. In a yet further aspect, the MET inhibitor is JNJ-38877605.

In a further aspect, the invention pertains to use of an effective amount of an MET inhibitor. In various aspects, an “effective amount” can be a therapeutically effective amount. In a further aspect, an “effective amount” can be a prophylactically effective amount.

Methods of preparing each of the disclosed MET inhibitors are known in to the skilled artisan.

### D. MEK INHIBITORS

The present invention pertains to the use of EGFR inhibitors. In various aspects, the MEK inhibitor is trametinib, binimetinib (MEK162), cobimetinib (GDC-0973), selumetinib (AZD6244), PD184161, BAY 86-9766, PD0325901, CI-1040, PD98059, PD318088, GSK 120212 (JTP-74057), AZD8330 (ARRY-424704), AZD6244 (ARRY-142886), ARRY-162, ARRY-300, AS703026, U0126, CH4987655, TAK-733, AS703026 (pimasertib, MSC1936369B), PD-325901, PD184352, or CI-1040 (PD184352), or combinations thereof. In a further aspect, the MEK inhibitor is trametinib, binimetinib (MEK162), cobimetinib (GDC-0973), selumetinib (AZD6244), or PD184161. In a still further aspect, the MEK inhibitor is trametinib. In a yet further aspect, the MEK inhibitor is PD184161. In an even further aspect, the MEK inhibitor is PD184161 or trametinib.

In a further aspect, the invention pertains to use of an effective amount of an MEK inhibitor. In various aspects, an “effective amount” can be a therapeutically effective amount. In a further aspect, an “effective amount” can be a prophylactically effective amount.

Methods of preparing each of the disclosed MEK inhibitors are known in to the skilled artisan.

### E. PHARMACEUTICAL COMPOSITIONS

In one aspect, the invention relates to pharmaceutical compositions comprising the disclosed EGFR inhibitors, the disclosed MET inhibitors, and/or the disclosed MEK inhibitors. That is, a pharmaceutical composition can be provided comprising an effective amount of at least one EGFR inhibitor, or a pharmaceutically acceptable salt thereof, and an effective amount of at least one disclosed MET inhibitor, or a pharmaceutically acceptable salt thereof. In various aspects, a pharmaceutical composition can be provided comprising an effective amount of at least one EGFR inhibitor, or a pharmaceutically acceptable salt thereof, and an effective amount of at least one disclosed MEK inhibitor, or a pharmaceutically acceptable salt thereof.

In one aspect, the invention pertains to pharmaceutical compositions comprising: (a) an effective amount of an EGFR inhibitor, or a pharmaceutically acceptable salt thereof; and (b) an effective amount of a MEK inhibitor or a MET inhibitor, or a combination thereof, or a pharmaceutically acceptable salt thereof; and a pharmaceutically acceptable carrier. The pharmaceutical composition can comparise any one or more of the EGFR inhibitors disclosed herein above. It is also understood that the pharmaceutical composition can comprise any one or more of the MEK or MET inhibitors disclosed herein above. In a further aspect, the pharmaceutical composition further comprises a therapeutic agent known to treat cancer. In a still further aspect, the pharmaceutical composition further comprises 5-fluorouracil, oxaliplatin, or leucovorin, or a combination thereof.

In various aspects, the effective amount of the pharmaceutical composition is a therapeutically effective amount. In a still further aspect, the effective amount of the pharmaceutical composition is a prophylactically effective amount. In a further aspect, the effective amount of the EGFR inhibitor is a therapeutically effective amount. In a still further aspect, the effective amount of the EGFR inhibitor is a prophylactically effective amount. In an even further aspect, the effective amount of the MEK inhibitor is a therapeutically effective amount. In a still further aspect, the effective amount of the MEK inhibitor is a prophylactically effective amount. In a yet further aspect, the effective amount of the MET inhibitor is a therapeutically effective amount. In an even further aspect, the effective amount of the MET inhibitor is a prophylactically effective amount.

In a further aspect, the invention pertains to pharmaceutical compositions comprising: (a) an effective amount ofgefitinib, or a pharmaceutically acceptable salt thereof; and (b) an effective amount of PD184161, or a combination thereof, or a pharmaceutically acceptable salt thereof; and a pharmaceutically acceptable carrier.

In a further aspect, the invention pertains to pharmaceutical compositions comprising: (a) an effective amount ofgefitinib, or a pharmaceutically acceptable salt thereof; and (b) an effective amount of trametinib, or a combination thereof, or a pharmaceutically acceptable salt thereof; and a pharmaceutically acceptable carrier.

In a further aspect, the invention pertains to pharmaceutical compositions comprising: (a) an effective amount of erlotinib, or a pharmaceutically acceptable salt thereof; and (b) an effective amount of PD184161, or a combination thereof, or a pharmaceutically acceptable salt thereof; and a pharmaceutically acceptable carrier.

In a further aspect, the invention pertains to pharmaceutical compositions comprising: (a) an effective amount of erlotinib, or a pharmaceutically acceptable salt thereof; and (b) an effective amount of trametinib, or a combination thereof, or a pharmaceutically acceptable salt thereof; and a pharmaceutically acceptable carrier.

In a further aspect, the invention pertains to pharmaceutical compositions comprising: (a) an effective amount of lapatinib, dacomitinib, neratinib, or a pharmaceutically acceptable salt thereof; and (b) an effective amount of PD184161, or a combination thereof, or a pharmaceutically acceptable salt thereof; and a pharmaceutically acceptable carrier.

In a further aspect, the invention pertains to pharmaceutical compositions comprising: (a) an effective amount of lapatinib, dacomitinib, neratinib, or a pharmaceutically acceptable salt thereof; and (b) an effective amount of trametinib, or a combination thereof, or a pharmaceutically acceptable salt thereof; and a pharmaceutically acceptable carrier.

In a further aspect, the invention pertains to pharmaceutical compositions comprising: (a) an effective amount of vandetanib, or a pharmaceutically acceptable salt thereof; and (b) an effective amount of PD184161, or a combination thereof, or a pharmaceutically acceptable salt thereof; and a pharmaceutically acceptable carrier.

In a further aspect, the invention pertains to pharmaceutical compositions comprising: (a) an effective amount of vandetanib, or a pharmaceutically acceptable salt thereof; and (b) an effective amount of trametinib, or a combination thereof, or a pharmaceutically acceptable salt thereof; and a pharmaceutically acceptable carrier.

In a further aspect, the invention pertains to pharmaceutical compositions comprising: (a) an effective amount of cetuximab, or a pharmaceutically acceptable salt thereof; and (b) an effective amount of PD184161, or a combination thereof, or a pharmaceutically acceptable salt thereof; and a pharmaceutically acceptable carrier.

In a further aspect, the invention pertains to pharmaceutical compositions comprising: (a) an effective amount of cetuximab, or a pharmaceutically acceptable salt thereof; and (b) an effective amount of trametinib, or a combination thereof, or a pharmaceutically acceptable salt thereof; and a pharmaceutically acceptable carrier.

In a further aspect, the invention pertains to pharmaceutical compositions comprising: (a) an effective amount of panitumumab, or a pharmaceutically acceptable salt thereof; and (b) an effective amount of PD184161, or a combination thereof, or a pharmaceutically acceptable salt thereof; and a pharmaceutically acceptable carrier.

In a further aspect, the invention pertains to pharmaceutical compositions comprising: (a) an effective amount of panitumumab, or a pharmaceutically acceptable salt thereof; and (b) an effective amount of trametinib, or a combination thereof, or a pharmaceutically acceptable salt thereof; and a pharmaceutically acceptable carrier.

In one aspect, the pharmaceutical composition is used to treat a mammal. In a yet further aspect, the mammal is a human. In a further aspect, the mammal has been diagnosed with a need for treatment of the disorder, e.g., a cancer or a cancer associated with an EGFR dysfunction, prior to the administering step. In a further aspect, the mammal has been identified to be in need of treatment of the disorder. In a further aspect, the pharmaceutical composition is used to treat a cancer. In a yet further aspect, the disorder is associated with an EGFR dysfunction.

In a further aspect, the pharmaceutical composition is used to treat a cancer. In a still further aspect, the pharmaceutical composition is used to treat a cancer associated with EGFR. In a yet further aspect, the pharmaceutical composition is used to treat a cancer such as colorectal cancer, lung cancer, pancreatic cancer, melanoma, breast cancer, or head/neck cancer. In an even further aspect, the pharmaceutical composition is used to treat colorectal cancer. In a still further aspect, the pharmaceutical composition is used to treat lung cancer. In a yet further aspect, the pharmaceutical composition is used to treat non-small cell lung cancer. In an even further aspect, the pharmaceutical composition is used to treat pancreatic cancer. In a still further aspect, the pharmaceutical composition is used to treat melanoma. In a yet further aspect, the pharmaceutical composition is used to treat breast cancer. In an even further aspect, the pharmaceutical composition is used to treat head/neck cancer.

In certain aspects, the disclosed pharmaceutical compositions comprise the disclosed EGFR inhibitors and the disclosed MEK or MET inhibitors (including pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.); and Remington's Pharmaceutical Sciences (16th ed. 1980, Mack Publ. Co. of Easton, Pa., U.S.A.), e.g., pp. 1553-1576 for methods of making tablets, capsules and pills and their respective components are described.

Suitable binders, lubricants, disintegrating agents, coloring agents, flavoring agents, flow-inducing agents, and melting agents may be included as carriers, e.g., for pills. For instance, an active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like.

Suitable binders include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, for example, starch, methyl cellulose, agar, bentonite, xanthan gum, and the like.

In practice, the disclosed EGFR inhibitors and the disclosed MEK or MET inhibitors, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention can include a pharmaceutically acceptable carrier and at least one disclosed EGFR inhibitor and at least one disclosed MEK or MET inhibitor of the invention. The compounds of the invention, or pharmaceutically acceptable salts thereof, can also be included in pharmaceutical compositions in combination with one or more other therapeutically active compounds.

The disclosed EGFR inhibitors and MET or MEK inhibitors can be administered orally in solid dosage forms, such as capsules, tablets, and powders, or in liquid dosage forms, such as elixirs, syrups, and suspensions. The active compounds can also be administered parenterally, in sterile liquid dosage forms.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, powder packets, wafers, suppositories, injectable solutions or suspensions and the like, and segregated multiples thereof.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In order to enhance the solubility and/or the stability of the compounds of Forxmula (I) in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the invention in pharmaceutical compositions.

In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques

A tablet containing the composition of this invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

Alternatively, an oral dosage form of the disclosed pharmaceutical compositions can be a liquid oral dosage form. Examples liquid forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Liquid dosage forms may contain, for example, suitable solvents, preservatives, emulsifying agents, suspending agents, diluents, sweeteners, thickeners, and melting agents.

Liquid dosage forms for oral administration can contain coloring and flavoring, as needed. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The pharmaceutical compositions of the present invention comprise at least one disclosed EGFR inhibitors and at least one disclosed MEK or MET inhibitor (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

The at least one disclosed EGFR inhibitors and at least one disclosed MEK or MET inhibitor described herein may also be administered in intranasal form via use of suitable intranasal vehicles, or via transdermal routes, using those forms of transdermal skin patches known to those skilled in these arts. To be administered in the form of a transdermal delivery system, the dosage administration will generally be continuous rather than intermittent throughout the dosage regimen. Parenteral and intravenous forms may also include minerals and other materials to make them compatible with the type of injection or delivery system chosen.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Formulations suitable for parenteral administration include aqueous and non-aqueous formulations isotonic with the blood of the intended recipient; and aqueous and non-aqueous sterile suspensions which may include suspending systems designed to target the compound to blood components of one or more organs. The formulations may be presented in unit-dose or multi-dose sealed containers, for example, ampoules or vials. Extemporaneous injection solutions and suspensions may be prepared from sterile powders, granules and tablets of the kind previously described. Parenteral and intravenous formulation may include minerals and other materials to make them compatible with the type of injection or delivery system chosen.

Commonly used pharmaceutically acceptable carriers for parenteral administration includes, water, a suitable oil, saline, aqueous dextrose (glucose), or related sugar solutions and glycols such as propylene glycol or polyethylene glycols. Solutions for parenteral administration preferably contain a water soluble salt of the active ingredient, suitable stabilizing agents and, if necessary, buffer substances, antioxidizing agents, such as sodium bisulfite, sodium sulfite, or ascorbic acid, either alone or combined, are suitable stabilizing agents. Citric acid salts and sodium EDTA may also be used as carriers. In addition, parenteral solutions may contain preservatives, such as benzalkonium chloride, methyl- or propyl-paraben, or chlorobutanol. Suitable pharmaceutical carriers are described in Remington, cited above.

Injectable formulations can be prepared in conventional forms, either as liquid solutions or suspensions; as solid forms suitable for solubilization or suspension in liquid prior to injection; or as emulsions. Preferably, sterile injectable suspensions are formulated according to techniques known in the art using suitable pharmaceutically acceptable carriers and other optional components, as described above.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Parenteral administration may be carried out in any number of ways, but it is preferred that a syringe, catheter, or similar device, be used to effect parenteral administration of the formulations described herein. The formulation may be injected systemically such that the active agent travels substantially throughout the entire bloodstream.

Also, the formulation may also be injected locally to a target site, e.g., injected to a specific portion of the body for which inhibition of a pathway using DNA repair or replication is desired. An advantage of local administration via injection is that it limits or avoids exposure of the entire body to the active agent(s) (e.g., inhibitors and/or other therapeutic agents). It must be noted that in the present context, the term local administration includes regional administration, e.g., administration of a formulation directed to a portion of the body through delivery to a blood vessel serving that body zone. Local delivery may be direct, e.g., intratumoral. Local delivery may also be nearly direct, i.e., intralesional or intraperitoneal, that is, to an area that is sufficiently close to a tumor or site of infection so that the inhibitor exhibits the desired pharmacological activity. Thus, when local delivery is desired, the pharmaceutical formulations are preferably delivered intralesionally, intratumorally, or intraperitoneally.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, and/or pharmaceutically acceptable salts thereof, can also be prepared in powder or liquid concentrate form.

The exact dosage and frequency of administration depends on the particular disclosed EGFR inhibitor, or pharmaceutically acceptable salt thereof, and the particular MEK or MET inhibitor, or pharmaceutically acceptable salt thereof; the particular cancer being treated and the severity of the cancer being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredients, and, from 1 to 99.95% by weight, preferably from 30 to 99.9% by weight, more preferably from 50 to 99.9% by weight of a pharmaceutically acceptable carrier, all percentages being based on the total weight of the composition.

In the treatment conditions which require inhibition of EGFR activity and inhibition of MET kinase activity or MEK kinase activity, an appropriate dosage level will generally be about 0.01 to 500 mg per kg patient body weight per day of each active ingredient and can be administered in single or multiple doses. Preferably, the dosage level will be about 0.1 to about 250 mg/kg per day; more preferably 0.5 to 100 mg/kg of each active ingredient per day. A suitable dosage level can be about 0.01 to 250 mg/kg per day, about 0.05 to 100 mg/kg per day, or about 0.1 to 50 mg/kg of each active ingredient per day. Within this range the dosage can be 0.05 to 0.5, 0.5 to 5.0 or 5.0 to 50 mg/kg per day of each active ingredient. For oral administration, the compositions are preferably provided in the form of tablets containing 1.0 to 1000 milligrams of the active ingredient, particularly 1.0, 5.0, 10, 15, 20, 25, 50, 75, 100, 150, 200, 250, 300, 400, 500, 600, 750, 800, 900 and 1000 milligrams of each active ingredient for the symptomatic adjustment of the dosage of the patient to be treated. The compound can be administered on a regimen of 1 to 4 times per day, preferably once or twice per day. This dosing regimen can be adjusted to provide the optimal therapeutic response.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy.

Such unit doses as described hereinabove and hereinafter can be administered more than once a day, for example, 2, 3, 4, 5 or 6 times a day. In various aspects, such unit doses can be administered 1 or 2 times per day, so that the total dosage for a 70 kg adult is in the range of 0.001 to about 15 mg per kg weight of subject per administration. In a further aspect, dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

In determining the effective dose or dosage of the pharmaceutical composition of the invention, a response to a prophylatic and/or treatment method of the invention can, for example, also be measured by determining the physiological effects of the treatment or medication, such as the decrease or lack of disease symptoms following administration of the treatment or pharmacological agent. Other assays will be known to one of ordinary skill in the art and can be employed for measuring the level of the response. For example, the behavioral and neurological diagnostic methods that are used to ascertain the likelihood that a subject has Alzheimer's disease, and to determine the putative stage of the disease can be used to ascertain the level of response to a prophylactic and/or treatment method of the invention. The amount of a treatment may be varied for example by increasing or decreasing the amount of a therapeutic composition, by changing the therapeutic composition administered, by changing the route of administration, by changing the dosage timing and so on. The effective amount will vary with the particular condition being treated, the age and physical condition of the subject being treated, the severity of the condition, the duration of the treatment, the nature of the concurrent therapy (if any), the specific route of administration, and the like factors within the knowledge and expertise of the health practitioner. For example, an effective amount can depend upon the degree to which an individual has abnormal levels and/or activity of a secretase pathway associated protein or secretase pathway associated protein complex.

The factors involved in determining an effective amount are well known to those of ordinary skill in the art and can be addressed with no more than routine experimentation. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

The therapeutically effective amount of the EGFR inhibitor and MEK or MET inhibitor of the invention is that amount effective to reduce, prevent, or eliminate a cancer-related malignancy. For example, testing can be performed to determine the level of cancerous cells in a tissue biopsy and/or serum HGF levels.

In the case of treating a particular disease or condition the desired response is inhibiting the progression of the disease or condition. This may involve only slowing the progression of the disease temporarily, although more preferably, it involves halting the progression of the disease permanently. This can be monitored by routine diagnostic methods known to one of ordinary skill in the art for any particular disease. The desired response to treatment of the disease or condition also can be delaying the onset or even preventing the onset of the disease or condition.

The present invention is further directed to a method for the manufacture of a medicament for treatment of cancer in mammals (e.g., humans) comprising combining one or more disclosed compounds, products, or compositions with a pharmaceutically acceptable carrier or diluent. Thus, in one aspect, the invention relates to a method for manufacturing a medicament comprising combining at least one disclosed compound or at least one disclosed product with a pharmaceutically acceptable carrier or diluent.

The disclosed pharmaceutical compositions can further comprise other therapeutically active compounds, which are usually applied in the treatment of the above mentioned pathological conditions.

It is understood that the disclosed compositions can be prepared from the disclosed compounds. It is also understood that the disclosed compositions can be employed in the disclosed methods of using.

The disclosed EGFR inhibitors and MET or MEK inhibitors may also be used with liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The disclosed EGFR inhibitors and MET or MEK inhibitors may also be coupled to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

In various aspects, the pharmaceutical compositions are in unit dosage form. As used herein, the term “unit dose” or “unit dosage” refers to a physically discrete unit that contains a predetermined quantity of active ingredient calculated to produce a desired therapeutic effect.

In such form, the composition is divided into unit doses containing appropriate quantities of the active ingredients. The unit dosage form can be a packaged preparation, the package containing discrete quantities of the preparations, for example, packeted tablets, capsules, and powders in vials or ampoules. The unit dosage form can also be a capsule, cachet, or tablet, or it can be the appropriate number of any of these packaged forms.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

The method of administration of the compounds set forth herein can be any suitable method that is effective in the treatment of the particular cancer or tumor type being treated. Treatment may be oral, rectal, topical, parenteral or intravenous administration or by injection into a tumor or cancer. The method of applying an effective amount also varies depending on the disorder or disease being treated. Parenteral treatment may be, e.g., by intravenous, subcutaneous, or intramuscular application of the compounds set forth herein, formulated with an appropriate carrier, additional cancer inhibiting compound or compounds or diluent to facilitate application.

### F. METHODS OF TREATING A CANCER ASSOCIATED WITH EGFR

In various aspects, the invention pertains to methods for the treatment of a cancer in a mammal comprising the step of co-administering to the mammal: (a) an effective amount of an EGFR inhibitor, or a pharmaceutically acceptable salt thereof; and (b) an effective amount of a MEK inhibitor or a MET inhibitor, or a combination thereof, or a pharmaceutically acceptable salt thereof. In a further aspect, co-administratering is simultaneous administration of the EGFR inhibitor and the MEK inhibitor or the MET inhibitor. In a still further aspect, co-administratering is sequential administration of the EGFR inhibitor and the MEK inhibitor or the MET inhibitor. In a yet further aspect, the co-administration of the EGFR inhibitor and the MEK inhibitor or the MET inhibitor is co-packaged dosage forms of each inhibitor. In an even further aspect, the co-administration of the EGFR inhibitor and the MEK inhibitor or the MET inhibitor is co-formulated dosage forms of each inhibitor. In a still further aspect, co-administering is sequential administration of the EGFR inhibitor and the MEK inhibitor or the MET inhibitor, wherein the EGFR inhibitor is administered first followed by administration of the MEK inhibitor or the MET inhibitor. In a yet further aspect, co-administering is sequential administration of the EGFR inhibitor and the MEK inhibitor or the MET inhibitor, wherein the MEK inhibitor or the MET inhibitor is administered first followed by administration of the EGFR inhibitor.

In a further aspect, the method uses any one or more of the disclosed EGFR inhibitors. In a still further aspect, the method uses any one or more of the disclosed MEK inhibitors. In a yet further aspect, the method uses any one or more of the disclosed MET inhibitors.

In a further aspect, the mammal is a human. In a still further aspect, the mammal has been diagnosed with a need for treatment of the cancer prior to the administering step. In a yet further aspect, the method further comprises the step of identifying a mammal in need of treatment of the cancer.

In a further aspect, the mammal has been diagnosed with increased HGF expression. In a still further aspect, diagnosed with increased HGF expression is diagnosed with increased serum HGF levels. In a yet further aspect, the serum HGF level is greater than about 1000 pg/ml. In an even further aspect, the serum HGF level is greater than about 1100 pg/ml. In a still further aspect, the serum HGF level is greater than about 1300 pg/ml. In a yet further aspect, the serum HGF level is from about 1000 pg/ml to about 4000 pg/ml. In an even further aspect, the serum HGF level is from about 1100 pg/ml to about 3500 pg/ml. In a still further aspect, the serum HGF level is from about 1300 pg/ml to about 3400 pg/ml.

In a further aspect, identifying the mammal with increased HGF levels is identifying HGF gene mutations. In a still further aspect, the HGF mutation is a HGF promoter mutation. In a yet further aspect, the HGF promoter mutation is a deoxyadenosine tract element mutation. In an even further aspect, the deoxyadenosine tract element mutation is a truncation mutation. In a still further aspect, the truncation mutation results in less than or equal to about 25 adenosine residues in the deoxyadenosine tract element.

In a further aspect, the mammal has been diagnosed as having a wild-type KRas gene and being resistant to treatment with an EGFR inhibitor. In a still further aspect, the mammal has been diagnosed as having colorectal cancer, having a wild-type KRas gene and being resistant to treatment with an EGFR inhibitor. In a yet further aspect, the method further comprises the step of identifying a mammal having a wild-type KRas gene and is resistant to treatment with an EGFR inhibitor. In an even further aspect, the method further comprises the step of identifying a mammal having colorectal cancer, having a mutant KRas gene and is resistant to treatment with an EGFR inhibitor.

In a further aspect, the mammal has been diagnosed as having a mutant KRas gene and being resistant to treatment with an EGFR inhibitor. In a still further aspect, the mammal has been diagnosed as having colorectal cancer, having a mutant KRas gene and being resistant to treatment with an EGFR inhibitor. In a further aspect, the method further comprises the step of identifying a mammal having a mutant KRas gene and is resistant to treatment with an EGFR inhibitor.

In a further aspect, the KRas mutation is in codon 2, 3, 4, 12, 13, 61, or 146. In a still further aspect, the KRas mutation is in codon 12, 13, or 61. In a yet further aspect, the KRas mutation is in exon 3 or exon 4. In an even further aspect, the KRas mutation is G12C, G12R, G12S, G12V, G12D, G12A, G12F, G13V/D, A59T, Q61E/K, Q61L/R/P, or Q61H.

In various aspects, the method is a method of treating a cancer, wherein the cancer is colorectal cancer, lung cancer, pancreatic cancer, melanoma, breast cancer, or head/neck cancer. In a further aspect, the method is a method of treating a cancer, wherein the cancer is colorectal cancer. In a still further aspect, the method is a method of treating a cancer, wherein the cancer is lung cancer. In a yet further aspect, the method is a method of treating a cancer, wherein the cancer is non-small cell lung cancer. In an even further aspect, the method is a method of treating a cancer, wherein the cancer is pancreatic cancer. In a still further aspect, the method is a method of treating a cancer, wherein the cancer is pancreatic cancer. In a yet further aspect, the method is a method of treating a cancer, wherein the cancer is melanoma. In an even further aspect, the method is a method of treating a cancer, wherein the cancer is breast cancer. In a still further aspect, the method is a method of treating a cancer, wherein the cancer is head/neck cancer.

In one aspect the invention pertains to methods for the treatment of colorectal cancer in a human comprising the step of administering to the human: (a) an effective amount of an EGFR inhibitor, or a pharmaceutically acceptable salt thereof; and (b) an effective amount of a MEK inhibitor or a MET inhibitor, or a combination thereof, or a pharmaceutically acceptable salt thereof. In a further aspect, the mammal has been diagnosed with colorectal cancer prior to the administering step. In a still further aspect, the method further comprises comprising the step of identifying a mammal in need of treatment of colorectal cancer.

In one aspect, the invention pertains to methods for the treatment of a cancer comprising the steps of: identifying a mammal with increased HGF levels; and administering the mammal with increased HGF levels: (a) an effective amount of an EGFR inhibitor, or a pharmaceutically acceptable salt thereof; and (b) an effective amount of a MEK inhibitor or a MET inhibitor, or a combination thereof, or a pharmaceutically acceptable salt thereof.

In one aspect, the invention pertains to methods for decreasing resistance of a cancer to treatment with an EGFR inhibitor: (a) identifying a patient with increase HGF expression; and (b) administering to the patient with increased HGF expression a MET inhibitor.

The EGFR inhibitor and the MEK inhibitor or the MET inhibitor can be co-administered, either in concomitant therapy or in a fixed combination.

In a further aspect, the method comprises co-administering to the mammal a therapeutically effective amount of an EGFR inhibitor and the MEK inhibitor or the MET inhibitor. In a still further aspect, the co-administration is administration in a substantially simultaneous manner. In a yet further aspect, the administration in a substantially simultaneous manner comprises a single dose form containing a fixed ratio of the compound and the antibacterial agent. In an even further aspect, the single dose form is a capsule or a tablet. In a still further aspect, the single dose form is an ampule for a single intravenous administration. In a yet further aspect, the co-administration is administration in a substantially sequential manner.

While the disclosed EGFR inhibitors and MEK or MET inhibitors described herein may be clinically preferable for use in certain types of cancer, they are also expected to be useful in the treatment of a variety of cancers including, but not limited to: carcinoma such as bladder, breast, colon, kidney, liver, lung, including small cell lung cancer, esophagus, gall-bladder, ovary, pancreas, stomach, cervix, thyroid, prostate, and skin, including squamous cell carcinoma; hematopoietic tumors of lymphoid lineage, including leukemia, acute lymphocitic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xenoderoma pigmentosum, keratoctanthoma, thyroid follicular cancer and Kaposi's sarcoma. According to another embodiment of the invention, compounds of the invention are directed to therapies for cell proliferative disorders, for example, Alzheimer's disease, viral infections, auto-immune diseases and neurodegenerative disorders.

Additional cancers that the disclosed pharmaceutical compositions or disclosed methods may be useful for treating are include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: non-small cell lung, bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia [acute and chronic], acute lymphoblastic leukemia, chronic lymphocytic leukemia, myeloproliferative diseases, multiple myeloma, myelodysplastic syndrome), Hodgkin's disease, non-Hodgkin's lymphoma [malignant lymphoma]; Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above-identified conditions.

### G. DETERMINATION OF KRAS MUTATIONS

KRas mutations can be determined using various methods known to one skilled in the art, e.g., the presence of mutations can be determined in a suitable sample using sequencing of PCR amplified target sequences, QPCR, hydridization methods and other methods known to the skilled artisan.

The samples used herein are any cell, body tissue, or body fluid sample obtained from a subject. In various aspects, the cell or tissue sample includes colorectal cells and/or is a colorectal cell or tissue sample. In a further aspect, the cell or tissue sample includes pancreatic cells and/or is a pancreatic cell or tissue sample. In a still further aspect, the cell or tissue sample includes breast cell and/or is a breast cell or tissue sample.

The biological sample can be a biopsy such as a tissue biopsy or tissue extract. In one aspect, the biological sample can be a cell-containing sample. Samples of tissue and/or cells for use in the various methods described herein can be obtained through standard methods. Samples can be surgical samples of any type of tissue or body fluid. Samples can be used directly or processed to facilitate analysis (e.g., paraffin embedding). Exemplary samples include a cell, a cell scraping, a cell extract, a blood sample, a cerebrospinal fluid sample, a tissue biopsy, including punch biopsy, a tumor biopsy, a bodily fluid, a tissue, or a tissue extract or other methods. Samples also can be cultured cells, tissues, or organs.

A biological sample can be obtained from a subject immediately prior (e.g., within 12 hours) to testing or optionally stored (e.g., frozen). Essentially any biological sample suspected of containing cancer cells may be used in for assessment of various markers. For example, the biological sample can be a tissue sample, such as a tissue biopsy sample, known or suspected of containing cancer cells. The biological sample may be derived from a tissue suspected of being the site of origin of a primary tumor. Alternatively, the biological sample may be derived from a tissue or other biological sample distinct from the suspected site of origin of a primary tumor in order to detect the presence of metastatic cancer cells in the tissue or sample that have escaped the site of origin of the primary tumor. In certain aspects, the biological sample is a tissue biopsy sample derived from cancer tissue. In other aspects, the biological sample tested is selected from the group consisting of a peripheral blood sample, biopsy sample, lavage sample, sputum sample, serum sample, lymph node sample, bone marrow sample, urine sample, and pleural effusion sample.

Exemplary methods to detect KRas mutations are described by Tan and Du (see C. Tan and X. Du, World J Gastroenterol. (2012) 18(37): 5171-5180). In various aspects, KRas mutations can also be determined using the FDA approved therascreen KRAS RGQ PCR Kit, which detects 7 KRAS gene mutations (Qiagen, Inc., Valencia, Calif., US). Additional methods to determine KRas include nucleic acid sequencing (dideoxy and pyrosequencing), real-time PCR with melt-curve analysis and allele-specific PCR with various modes used to distinguish mutant from wild-type sequences (Lang A. H., et al., J. Mol. Diagn. (2011) 13, 23-28; Ogino S., et al., J. Mol. Diagn. (2005) 7, 413-421; Tsiatis A. C., J. Mol. Diagn. (2010) 12, 425-432; Pritchard C. C., et al., (2010) BMC Clin. Pathol. 10, 1-10; and Samowitz W., et al., Cancer Epidemiol. Biomarkers Prev. (2000) 9, 1193-1197).

Testing for mutations, e.g., in codons 12 or 13 of the KRAS gene, can be performed on formalin-fixed, paraffin-embedded tissue from the primary tumor or a metastasis using a variety of methods. BRAF V600E mutation status is determined via PCR amplification and DNA sequence analysis or allele-specific PCR on formalin-fixed, paraffin-embedded tissue from the primary tumor or a metastasis.

### H. MANUFACTURE OF A MEDICAMENT

In one aspect, the invention relates to a medicament comprising one or more disclosed EGFR inhibitors, or a pharmaceutically acceptable salt thereof, and one or more disclosed MEK inhibitors, or a pharmaceutically acceptable salt thereof, or one or more disclosed MET inhibitors, or a pharmaceutically acceptable salt thereof, or a combination of MEK and MET inhibitors, or a pharmaceutically acceptable salts thereof.

### I. USE OF THE PHARMACEUTICAL COMPOSITION

Also provided are the uses of the disclosed EGFR inhibitors and disclosed MEK or MET inhibitor. In various aspects, the use comprises use of an effective amount of at least one disclosed EGFR inhibitors, or a pharmaceutically acceptable salt thereof, and an effective amount of at least one disclosed MEK inhibitors, or a pharmaceutically acceptable salt thereof, or an effective amount of at least one disclosed MET inhibitors, or a pharmaceutically acceptable salt thereof, or an effective amount of a combination of disclosed MEK and MET inhibitors, or a pharmaceutically acceptable salts thereof. In a still further aspect, an effective amount is a therapeutically effective amount. In a yet further aspect, an effective amount is a prophylactically effective amount.

In a further aspect, the use relates to a process for preparing a pharmaceutical composition comprising a therapeutically effective amount of at least one disclosed EGFR inhibitors, or a pharmaceutically acceptable salt thereof, and a therapeutically effective amount of at least one disclosed MEK inhibitors, or a pharmaceutically acceptable salt thereof, or a therapeutically effective amount of at least one disclosed MET inhibitors, or a pharmaceutically acceptable salt thereof, or a therapeutically effective amount of a combination of disclosed MEK and MET inhibitors, or a pharmaceutically acceptable salts thereof, for use as a medicament.

In a further aspect, the use relates to a process for preparing a pharmaceutical composition comprising a therapeutically effective amount of at least one disclosed EGFR inhibitors, or a pharmaceutically acceptable salt thereof, and a therapeutically effective amount of at least one disclosed MEK inhibitors, or a pharmaceutically acceptable salt thereof, or a therapeutically effective amount of at least one disclosed MET inhibitors, or a pharmaceutically acceptable salt thereof, or a therapeutically effective amount of a combination of disclosed MEK and MET inhibitors, or a pharmaceutically acceptable salts thereof, wherein a pharmaceutically acceptable carrier is intimately mixed with a therapeutically effective amount of the EGFR inhibitor and MET or MEK inhibitor.

In various aspects, the use relates to a treatment of a cancer in a mammal. Also disclosed is the use of a disclosed EGFR inhibitor in combination with a MET or MEK inhibitor. In one aspect, the use is characterized in that the mammal is a human. In one aspect, the use is characterized in that the disorder is a cancer associated with an EGFR dysfunction.

In a further aspect, the use relates to the manufacture of a medicament for the treatment of a disorder associated with a muscarinic acetylcholine receptor dysfunction in a mammal. In a further aspect, the medicament is used in the treatment of a neurological and/or psychiatric disorder associated with a muscarinic acetylcholine receptor dysfunction in a mammal.

In a further aspect, the use relates to treating a cancer. In a still further aspect, the cancer is colorectal cancer, lung cancer, pancreatic cancer, melanoma, or head/neck cancer. In a yet further aspect, the cancer is colorectal cancer. In an even further aspect, the cancer is lung cancer. In a still further aspect, the lung cancer is non-small cell lung cancer (NSCLC).

In a further aspect, the use relates to co-administering to a patient a medicament comprising a disclosed EGFR inhibitor and a disclosed MET or MEK inhibitor, wherein the patient has been diagnosed as having a cancer. In a still further aspect, the patient has been diagnosed as having a colorectal cancer, lung cancer, pancreatic cancer, melanoma, or head/neck cancer. In a yet further aspect, the patient has been diagnosed as having colorectal cancer. In an even further aspect, the patient has been diagnosed as having lung cancer. In a still further aspect, the patient has been diagnosed as having non-small cell lung cancer (NSCLC).

It is understood that the disclosed uses can be employed in connection with the disclosed compounds, methods, compositions, and kits.

### J. KITS

The compounds set forth herein may also be used in pharmaceutical kits for the treatment of cancer, or other purposes, which comprise one or more containers containing a pharmaceutical composition comprising a therapeutically effective amount of the compound. Such kits may further include, if desired, one or more of various components, such as, for example, containers with the compound, containers with one or more pharmaceutically acceptable carriers, additional containers, and instructions. The instructions may be in printed or electronic form provided, for example, as inserts or labels, indicating quantities of the components to be administered, guidelines for administration, and/or guidelines for mixing the components.

In one aspect, the invention pertains to kits comprising: (a) an effective amount of an EGFR inhibitor, or a pharmaceutically acceptable salt thereof; and (b) an effective amount of a MEK inhibitor or a MET inhibitor, or a combination thereof, or a pharmaceutically acceptable salt thereof.

In various further aspects, the kit comprises an effective amount of the EGFR inhibitor, or a pharmaceutically acceptable salt thereof; and an effective amount of the MEK inhibitor or the MET inhibitor, or a combination thereof, or a pharmaceutically acceptable salt thereof. In a still further aspect, an effective amount is a therapeutically effective amount. In a yet further aspect, an effective amount is a prophylactically effective amount.

In various aspects, the EGFR inhibitor and the MEK inhibitor or the MET inhibitor, or a combination thereof, are co-formulated or co-packaged. In a further aspect, the EGFR inhibitor and the MEK inhibitor or the MET inhibitor, or a combination thereof, are co-formulated. In a still further aspect, the EGFR inhibitor and the MEK inhibitor or the MET inhibitor, or a combination thereof, co-packaged.

The kits can also a EGFR inhibitor and a MEK inhibitor or a MET inhibitor co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

The EGFR inhibitor used in the kit can be any one or more of the EGFR inhibitors disclosed herein. The MEK inhibitor used in the kit can be any one or more of the MEK inhibitors disclosed herein. The MET inhibitor used in the kit can be any one or more of the MET inhibitors disclosed herein.

In various aspects, the kit further comprises a plurality of dosage forms, the plurality comprising one or more doses; wherein each dose comprises an effective amount of the EGFR inhibitor and an effective amount of a MEK inhibitor or a MET inhibitor. In a still further aspect, the plurality of dosage forms are formulated for oral administration and/or intravenous administration. In a yet further aspect, the plurality of dosage forms are formulated for oral administration. In an even further aspect, the plurality of dosage forms are formulated for intravenous administration. In a still further aspect, the dosage form for the EGFR is formulated for intravenous administration and the dosage form for the MEK inhibor or the MEK inhibitor is formulated for oral administration. In a yet further aspect, the dosage form for the EGFR is formulated for oral administration and the dosage form for the MEK inhibor or the MEK inhibitor is formulated for intravenous administration.

In various aspects, the kit further comprises an effective amount of at least one agent known to treat a cancer. In a further aspect, the effective amount of the agent is a therapeutically effective amount. In a still further aspect, the agent comprises 5-fluorouracil, oxaliplatin, or leucovorin, or a combination thereof.

In a further aspect, the kit further comprises an effective amount of at least one agent known to treat a cancer and instructions for administering the agent. In a still further aspect, kit further comprises an effective amount of at least one agent known to treat a cancer, instructions for administering the agent, and instructions for administering the agent with the EGFR inhibitor and the MEK inhibitor or the MET inhibitor.

In a further aspect, the kit further comprises instructions for administering the agent with the EGFR inhibitor and the MEK inhibitor or the MET inhibitor. In a still further aspect, the kit further comprises instructions for administering the EGFR inhibitor and the MEK inhibitor. In a yet further aspect, the kit further comprises instructions for administering the EGFR inhibitor and the MET inhibitor.

In various aspects, the kit further comprises instructions for treating a cancer associated with an EGFR dysfunction.

It is understood that the disclosed kits can be prepared from the disclosed EGFR inhibitors and the disclosed MEK inhibitors and/or the MET inhibitors. It is also understood that the disclosed kits can be employed in connection with the disclosed methods of using.

### K. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Materials and Methods

a. Materials

Gefinitinib was purchased from Selleck Chemicals, LLC (Boston, Mass.); cetuximab was a gift from Dr. Sanjay Goel (Albert Einstein Cancer Center); the MET kinase inhibitor, JNJ 38877605, was obtained from Selleck Chemicals, LLC; and the MEKI kinase inhibitor, and PD184161, was obtained from Sigma-Aldrich Corporation (Saint Louis, Mo.). Cell culture media was obtained from Thermo Fisher Scientific Corporation (Carlsbad, Calif.). RKO cells, HT29 cells, HCT116 cells, Caco-2 cells, and CCD-18Co fibroblast cells were obtained from ATCC (Manassas, Va.). DiFi cells were a gift from Dr. Robert Coffey, Vandebilt University.

b. Statistical Analysis

All experiments were repeated at least three times. Statistical analysis was carried out using the Student's t test in the GraphPad Prism 5.0 software (GraphPad Software, Inc., La Jolla, Calif., US). Values with p<0.05 were considered statistically significant.

c. Cell Culture Methods

Caco-2 and CCD-18Co fibroblasts were maintained in in minimum essential medium (MEM), supplemented with 10% fetal bovine serum, L-glutamine and antibiotics; and HT29 cells were maintained in DMEM medium, supplemented with 10% FBS under standard culture conditions.

d. Viability and Clonogenic Assays

Cell viability assays were conducted using the CellTiter-Glo® Luminescent Cell Viability Assay kit purchased from Promega Corporation (Madison, Wis., US) and used per the manufacturer's protocol. Cells were seeded in 96-well plates at a density of 1×10⁴ cells per well and cell viability was assessed at the indicated time points.

For clonogenic assay cells were seeded in 6-well plates at a density of 400 cells per well and were treated as indicated. After colony formation (8-12 days), cells were stained with crystal violet (0.5% w/v) (see Kueng W., et al., Anal Biochem (1989) 182: 16-19), and the number of colonies was determined using ImageQuant TL software (GE Healthcare Life Sciences, Pittsburgh, Pa., US).

e. Caspase 3/7 Activity Assay

CCD-18Co fibroblasts were cultured in complete media containing 10% FBS until they formed confluent monoleyers. Media were removed and replaced with serum-free MEM. After 48 hours, supematants were collected, centrifuged and filtered through a 0.2 micron filter to remove cell debris. Conditioned media were used immediately or were aliquoted and stored at −80° C. DiFi cells (20,000 cells/well) were treated with gefitinib (0.5 μM) in the presence or the absence of conditioned medium from HGF-producing CCD-18Co (ATCC® CRL-1459™) fibroblasts (50%) and JNJ38807605. Thus, DiFi cells were cultured in their regular media or in the presence of a 1:1 solution of their normal growth media and the conditioned media). Caspase activity was assessed 18 hours after treatment using the Caspase-Glo® 3/7 Assay kit purchased from Promega Corporation (Madison, Wis., US) and used per the manufacturer's protocol.

f. Western Blotting Methods

Immunoblotting was performed using standard procedures. Nitrocellulose membranes (GE Healthcare Life Sciences, Pittsburgh, Pa.) were blocked with 5% non-fat milk for 1 hour at room temperature, and incubated with primary antibodies overnight at 4° C. Primary antibodies used were anti-p-ERK1/2 (Cell Signaling Technology, Inc., Danvers, Mass., US) and anti-β-actin (Sigma-Aldrich Co. LLC, Saint Louis, Mo., US). Following washing with TBS-T buffer, membranes were incubated with horseradish peroxidase-conjugated secondary antibodies for 1 hour and then washed with TBS-T. Immunoblots were developed using the Amersham Western Blotting ECL chemiluminescent reagents (GE Healthcare Life Sciences, Pittsburgh, Pa., US). Protein loading was normalized by probing blots for the expression of β-actin.

2. HGF/MET Signaling Mediates Resistance to EGFR Inhibitors (EGFRI) in Colon Cancer Cells with Wild-Type KRas (WT KRas).

The effect of treatment with gefitinib alone or in combination with an inhibitor of MET kinase activity (using the inhibitor JNJ388777605) or inhibitor of MEK kinase activity (using the inhibitor PD184161) on viability and clonogenic growth was determined in RKO and HT29 cells. Both RKO cells and HT29 cells have WT KRas, but RKO cells express HGF, whereas HT29 cells do not express HGF.

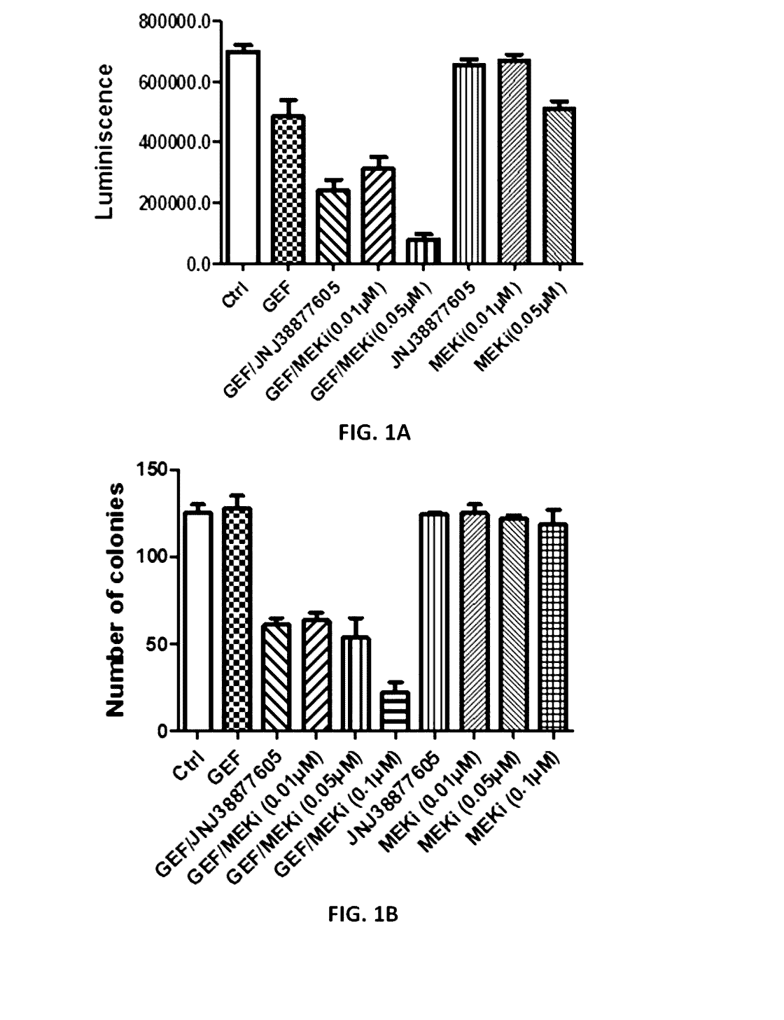

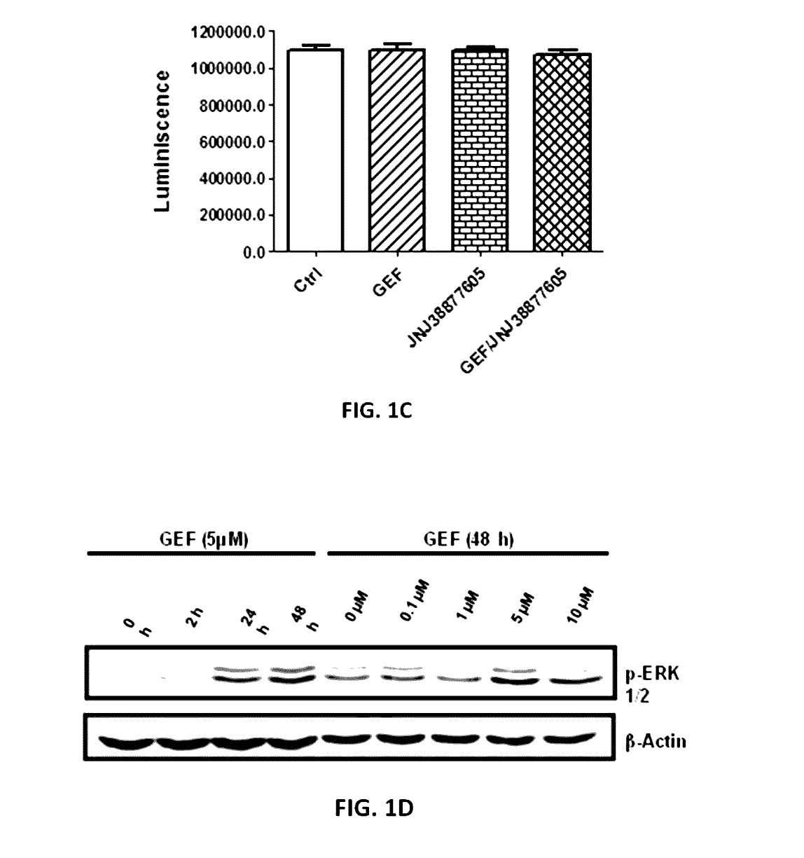

The data in FIG. 1A show that inhibition of MET kinase activity or inhibition of MEK kinase activity synergistically enhanced gefitinib-induced cell death in RKO cells. There was no apparent effect on cell viability when cells were exposed to only a MET kinase inhibitor or MEK kinase inhibitor. The effect of gefitinib alone in reducing viability of RKO cells was modest. The data in FIG. 1B show similar results for the effect on clonogenic growth of RKO cells. The data show that gefitinib in combination with a MET kinase inhibitor or in combination with a MEK inhibitor synergistically inhibited clonogenic growth. In contrast, treatment of RKO cells with gefitinib alone, MET kinase inhibitor alone, or MEK kinase inhibitor alone had no apparent effect on clonogenic growth of RKO cells.

In contrast to the results shown in FIGS. 1A and 1B, the data in FIG. 1C show that treatment of HT29 cells (which do not produce HGF) with a combination of gefitinib and a MET kinase inhibitor showed no improvement compared to treatment with gefitinib in the absence of a MET kinase inhibitor, indicating that a selected group of patients that overproduce HGF can benefit from combined treatment.

Without wishing to be bound by a particular model, these data considered together suggest that overproduction of HGF can cause a primary resistance to EGFR inhibitors in cells which have a WT KRas gene status. Accordingly, it is believed that patients with increased levels of HGF who have a WT KRas gene status would benefit from treatment with a combination therapeutic comprising an EGFR inhibitor and a therapeutic agent that can inhibit HGF/MET signaling, such as a MET kinase inhibitors.

The data in FIG. 1D show that treatment of RKO cells with gefitinib triggered a pro-survival ERK signaling in cancer cells (i.e., an increase in the amount of p-ERK 1/2 detected by Western blot) which are normally resistant to treatment with a EGFR inhibitor such as gefitinib. Inhibitors of EGFR have been shown to induce HGF in vitro and in cancer patients (Obenauf A. C., et al., Nature (2015) 520:368-72.), which acts as a potent inducer of ERK signaling.

3. HGF/MET Signaling Mediates Resistance to EGFR Inhibitors (EGFRI) in Colon Cancer Cells with Mutant KRas (MT KRas).

HCT116 cells, like RKO cells, express HGF (Seneviratne D. et al., Gastroenterology (2015) 148:181-91), but carry MT KRas, and do not respond to EGFR inhibitor treatment. Due to the presence of MT KRas, HCT116 cells have activated ERK signaling.

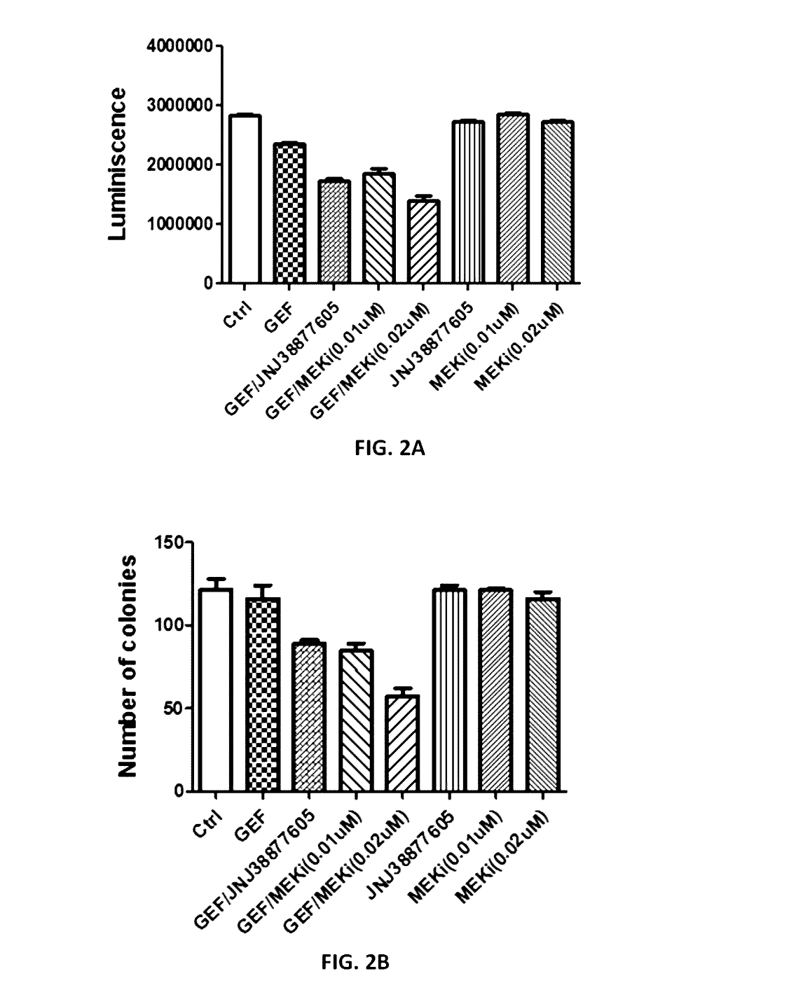

The data in FIG. 2A show that treatment of HCT116 cells with a combination of gefitinib and either an inhibitor of MET kinase activity (using the inhibitor JNJ388777605) or inhibitor of MEK kinase activity (using the inhibitor PD 184161) synergistically decreased cell viability. Treatment of HCT116 cells with gefitinib alone modestly decreased cell viability, whereas treatment with either the MET kinase inhibitor alone or the MEK kinase inhibitor alone had no apparent effect on cell viability.

The data in FIG. 2B show similar results for the effect on clonogenic growth. In particular, the treatment of HCT 116 cells with a combination of gefitinib and either an inhibitor of MET kinase activity (using the inhibitor JNJ388777605) or inhibitor of MEK kinase activity (using the inhibitor PD 184161) synergistically decreased clonogenic growth. Treatment of HCT116 cells with gefitinib alone, the MET kinase inhibitor alone, or the MEK kinase inhibitor alone had no apparent effect on clonogenic growth.

Without wishing to be bound by a particular theory, these data—in FIGS. 1A-ID and FIGS. 2A-2B—suggest that autocrine production of HGF underlies primary resistance to EGFR inhibitors in colon cancer cells irrespective of the KRas status. In addition, therapy-induced ERK activation or constitutive ERK signaling contribute to resistance to EGFRi. These findings have important implications for the treatment of colon cancer patients in general, but specifically for patients with MT KRas who have limited treatment options. Moreover, serum levels of HGF may constitute a strategy to select colon cancer patients for combined anti-MET and anti-EGFR therapy.

4. Inhibition of HGF/MET Signaling Prevents the Tumor Microenvironment-Mediated Resistance to EGFR Inhibitors.

Resistance to targeted therapy often originates from the tumor microenvironment, with cancer associated fibroblasts being a major source of HGF (Klemm F. and Joyce J. A., Trends in Cell Biol. (2015) 25:198-213; and Junttila M. R. and de Sauvage F. J. Nature (2013) 501:346-54).

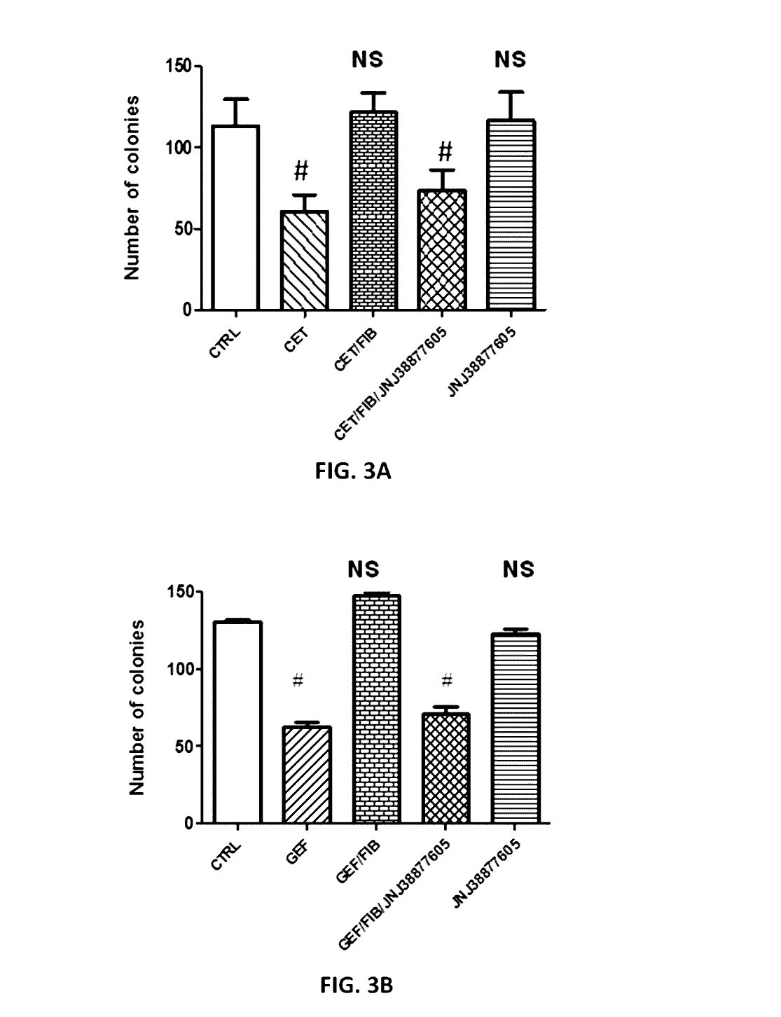

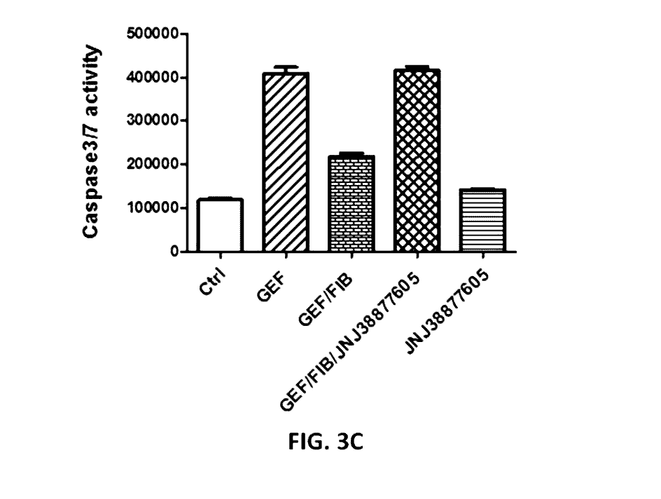

FIG. 3A shows data for the effect of an inhibitor of MET kinase activity (using the inhibitor JNJ388777605) on sensitivity of Caco2 cells to an EGFR inhibitor, cetuximab, when the cells were also exposed to conditioned media from fibroblast cells expressing HGF. The data show that fibroblast-derived factors completely inhibit the response of CaCo2 cells to cetuximab. Sensitivity was restored when these cells were treated with a combination of cetuximab and the MET kinase inhibitor, demonstrating that fibroblasts inhibit the response to cetuximab through HGF production. Treatment of Caco2 cells with JNJ3887765 alone did not impact their clonogenic growth.

Similar results were seen using the gefitinib, an EGFR kinase inhibitor. FIG. 3B shows data for the effect of an inhibitor of MET kinase activity (using the inhibitor JNJ388777605) on sensitivity of clonogenic Caco2 cells to an EGFR kinase inhibitor, gefitinib, when the cells were also exposed to conditioned media from fibroblast cells expressing HGF. The data established that fibroblasts inhibit the response to gefitinib. Sensitivity was restored when these cells were treated with a combination ofgefitinib and the MET kinase inhibitor. Caco2 cells did not respond to treatment with the MET kinase inhibitor alone.

The data in FIG. 3C show caspase 3/7 activity, a reliable measure of apoptosis, in DiFi cells treated with gefitinib (1 μM) in the absence or presence of conditioned media from fibroblasts (FIB) and a MET kinase inhibitor (JNJ 38877605; 1 μM) as indicated at 18 hr post-treatment. DiFi colon cancer cells undergo apoptosis in response to inhibition of EGFR, which is shown by the increase in caspase 3/7 activity (FIG. 3C, “GEF” data). Condition media from CCD-18Co fibroblast cells inhibit gefitinib-induced apoptosis in Caco-2 cells (FIG. 3C, “GEF/FIB” data). However, the data show that inhibition of the MET kinase activity by JNJ 38877605 restored apoptosis in response to EGFR inhibition (FIG. 3C, “GEF/FIB/JNJ38877605”), demonstrating that fibroblasts inhibit gefitinib induced caspase activity via HGF production. Treatment of Caco-2 cells with JN38877605 alone also has no apparent effect on induction of caspases/apoptosis (FIG. 3C, “JNJ3887065”).

Without wishing to be bound by a particular theory, these data suggest that HGF is a major source of the tumor microenvironment-mediated resistance to EGFR inhibitors and that inhibition of HGF/MET signaling can restore the response of cancer cells to EGFR inhibitors.

5. Prophetic Pharmaceutical Composition Examples

“First active ingredient” as used throughout these examples relates to one or more of the EGFR inhibitors as described hereinbefore, or a pharmaceutically acceptable salt thereof. “Second active ingredient” as used throughout these examples relates to either one or more of the MET inhibitors or one or more of the MEK inhibitors as described hereinbefore, or a pharmaceutically acceptable salt thereof. The following examples of the formulation of the compounds of the present invention in tablets, suspension, injectables and ointments are prophetic. Typical examples of recipes for the formulation of the invention are as given below.

Typical examples of recipes for the formulation of the invention are as given below. Various other dosage forms can be applied herein such as a filled gelatin capsule, liquid emulsion/suspension, ointments, suppositories or chewable tablet form employing the disclosed compounds in desired dosage amounts in accordance with the present invention. Various conventional techniques for preparing suitable dosage forms can be used to prepare the prophetic pharmaceutical compositions, such as those disclosed herein and in standard reference texts, for example the British and US Pharmacopoeias, Remington's Pharmaceutical Sciences (Mack Publishing Co.) and Martindale The Extra Pharmacopoeia (London The Pharmaceutical Press).

The disclosure of this reference is hereby incorporated herein by reference.

a. Pharmaceutical Composition for Oral Administration

A tablet can be prepared as follows:

| Component | Amount | |
|---|---|---|
| First active ingredient | 10 to 500 | mg |
| Second active ingredient | 10 to 500 | mg |
| Lactose | 100 | mg |
| Crystalline cellulose | 60 | mg |
| Magnesium stearate | 5 | |
| Starch (e.g. potato starch) | Amount necessary to yield total | |
| | weight indicated below | |
| Total (per capsule) | 1000 | mg |

Alternatively, about 100-250 mg of a first active ingredient, 100-250 meg of a second active ingredient, 50 mg of lactose (monohydrate), 50 mg of maize starch (native), 10 mg of polyvinylpyrrolidone (PVP 25) (e.g. from BASF, Ludwigshafen, Germany) and 2 mg of magnesium stearate are used per tablet. The mixture of active component, lactose and starch is granulated with a 5% solution (m/m) of the PVP in water. After drying, the granules are mixed with magnesium stearate for 5 min. This mixture is moulded using a customary tablet press (e.g. tablet format: diameter 8 mm, curvature radius 12 mm). The moulding force applied is typically about 15 kN.

Alternatively, a disclosed compound can be administered in a suspension formulated for oral use. For example, about 50-500 mg of the first active ingredient, 50-500 mg of the second active ingredient, 1000 mg of ethanol (96%), 400 mg of xanthan gum, and 99 g of water are combined with stirring. A single dose of about 10-500 mg of the desired disclosed compound according can be provided by 10 ml of oral suspension.

In some circumstances it may be desirable to use a capsule, e.g. a filled gelatin capsule, instead of a tablet form. The choice of tablet or capsule will depend, in part, upon physicochemical characteristics of the particular disclosed compound used.

Examples of alternative useful carriers for making oral preparations are lactose, sucrose, starch, talc, magnesium stearate, crystalline cellulose, methyl cellulose, hydroxypropyl cellulose, hydroxypropylmethyl cellulose, carboxymethyl cellulose, glycerin, sodium alginate, gum arabic, etc. These alternative carriers can be substituted for those given above as required for desired dissolution, absorption, and manufacturing characteristics.

The amount of a disclosed compound per tablet for use in a pharmaceutical composition for human use is determined from both toxicological and pharmacokinetic data obtained in suitable animal models, e.g. rat and at least one non-rodent species, and adjusted based upon human clinical trial data. For example, it could be appropriate that a disclosed compound is present at a level of about 10 to 1000 mg per tablet dosage unit.

b. Pharmaceutical Composition for Injectable Use

A parenteral composition can be prepared as follows:

| Component | Amount | |
|---|---|---|
| First active ingredient | 10 to 500 | mg |
| Second active ingredient | 10 to 500 | mg |
| Sodium carbonate | 560 | mg* |
| Sodium hydroxide | 80 | mg* |
| Distilled, sterile water | Quantity sufficient to prepare | |
| | total volume indicated below. | |
| Total (per capsule) | 10 | ml per ampule |

*Amount adjusted as required to maintain physiological pH in the context of the amount of active ingredient, and form of active ingredient, e.g. a particular salt form of the active ingredient.

Alternatively, a pharmaceutical composition for intravenous injection can be used, with composition comprising about 50-500 mg of the first active ingredient, 50-500 mg of the second active ingredient, 15 g polyethylenglycol 400 and 250 g water in saline with optionally up to about 15% Cremophor EL, and optionally up to 15% ethyl alcohol, and optionally up to 2 equivalents of a pharmaceutically suitable acid such as citric acid or hydrochloric acid are used. The preparation of such an injectable composition can be accomplished as follows: The disclosed compound and the polyethylenglycol 400 are dissolved in the water with stirring. The solution is sterile filtered (pore size 0.22 μn) and filled into heat sterilized infusion bottles under aseptic conditions. The infusion bottles are sealed with rubber seals.

In a further example, a pharmaceutical composition for intravenous injection can be used, with composition comprising about 50-500 mg of the first active ingredient, 50-500 mg of the second active ingredient, standard saline solution, optionally with up to 15% by weight of Cremophor EL, and optionally up to 15% by weight of ethyl alcohol, and optionally up to 2 equivalents of a pharmaceutically suitable acid such as citric acid or hydrochloric acid. Preparation can be accomplished as follows: a desired disclosed compound is dissolved in the saline solution with stirring. Optionally Cremophor EL, ethyl alcohol or acid are added. The solution is sterile filtered (pore size 0.22 μm) and filled into heat sterilized infusion bottles under aseptic conditions. The infusion bottles are sealed with rubber seals.

In this example, active ingredient can be replaced with the same amount of any of the compounds according to the present invention, in particular by the same amount of any of the exemplified compounds.

The amount of a disclosed compound per ampule for use in a pharmaceutical composition for human use is determined from both toxicological and pharmacokinetic data obtained in suitable animal models, e.g. rat and at least one non-rodent species, and adjusted based upon human clinical trial data. For example, it could be appropriate that a disclosed compound is present at a level of about 10 to 1000 mg per tablet dosage unit.

Carriers suitable for parenteral preparations are, for example, water, physiological saline solution, etc. which can be used with tris(hydroxymethyl)aminomethane, sodium carbonate, sodium hydroxide or the like serving as a solubilizer or pH adjusting agent. The parenteral preparations contain preferably 50 to 1000 mg of a disclosed compound per dosage unit.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A pharmaceutical composition comprising:
a) an effective amount of an EGFR inhibitor, or a pharmaceutically acceptable salt thereof; and
b) an effective amount of a MEK inhibitor or a MET inhibitor, or a combination thereof, or a pharmaceutically acceptable salt thereof;
and a pharmaceutically acceptable carrier.

2. The composition of claim 1, wherein the EGFR inhibitor is gefitinib, erlotinib, afatinib, rociletinib, vandetanib, dacomitinib, neratinib, lapatinib, icotinib, ibrutinib, cetuximab, panitumumab, zalutumumab, nimtuzumab, or matuzumab, or combinations thereof.

3. The composition of claim 1, wherein the EGFR inhibitor is gefitinib.

4. The composition of claim 1, wherein the MEK inhibitor is trametinib, binimetinib (MEK162), cobimetinib (GDC-0973), selumetinib (AZD6244), PD184161, BAY 86-9766, PD0325901, CI-1040, PD98059, PD318088, GSK 120212 (JTP-74057), AZD8330 (ARRY-424704), AZD6244 (ARRY-142886), ARRY-162, ARRY-300, AS703026, U0126, CH4987655, TAK-733, AS703026 (pimasertib, MSC1936369B), PD-325901, PD 184352, or CI-1040 (PD184352), or combinations thereof.

5. The composition of claim 1, wherein the MEK inhibitor is trametinib or PD184161.

6. The composition of claim 1, wherein the MET inhibitor is crizotinib, cabozantinib, tivantinib, foretinib, golvatinib, JNJ-38877605, PHA-665752, SU11274, SGX-523, PF-04217903, EMD 1214063, INCB28060, MK-2461, NVP-BVU972, AMG458, BMS 794833, BMS 777607, MGCD-265, AMG-208, or BMS-754807, or combinations thereof.

7. The composition of claim 1, wherein the MET inhibitor is JNJ-38877605.

8. The composition of claim 1, wherein the EGFR inhibitor is gefitinib; and wherein the MEK inhibitor is PD184161.

9. The composition of claim 1, wherein the EGFR inhibitor is gefitinib; and wherein the MET inhibitor is JNJ-38877605.

10. The composition of claim 1, further comprising 5-fluorouracil, oxaliplatin, or leucovorin, or a combination thereof.

11. A method for the treatment of a cancer, the method comprising the steps of:
identifying a mammal with increased HGF levels; and
administering to the mammal with increased HGF levels:
a) an effective amount of an EGFR inhibitor, or a pharmaceutically acceptable salt thereof; and
b) an effective amount of a MEK inhibitor or a MET inhibitor, or a combination thereof, or a pharmaceutically acceptable salt thereof.

12. The method of claim 11, wherein identifying the mammal with increased HGF levels is determining the level of serum HGF.

13. The method of claim 12, wherein the serum HGF level is greater than about 1300 pg/ml.

14. The method of claim 11, wherein identifying the mammal with increased HGF levels is identifying a HGF gene mutation.

15. The method of claim 14, wherein the HGF mutation is a HGF promoter mutation; and wherein the HGF promoter mutation is a deoxyadenosine tract element truncation mutation.

16. The method of claim 15, wherein the truncation mutation results in less than or equal to about 25 adenosine residues in the deoxyadenosine tract element.

17. The method of claim 11, wherein the cancer is colorectal cancer, lung cancer, pancreatic cancer, melanoma, breast cancer, or head/neck cancer.

18. A kit comprising:
a) an effective amount of an EGFR inhibitor, or a pharmaceutically acceptable salt thereof; and
b) an effective amount of a MEK inhibitor or a MET inhibitor, or a combination thereof, or a pharmaceutically acceptable salt thereof.

19. The kit of claim 18, wherein the EGFR inhibitor and the MEK inhibitor are co-formulated or co-packaged.

20. The kit of claim 18, wherein the EGFR inhibitor and the MET inhibitor are co-formulated or co-packaged.
